(19) United States
(12) Patent Application Publication
(10) Pub. No.: US 2026/0216393 A1
(43) Pub. Date: Jul. 30, 2026

(54) FLUOROPOLYMER BIOPROCESS BAG

(71) Applicants: DAIKIN AMERICA INC., Orangeburg, NY (US); DAIKIN INDUSTRIES, LTD., Osaka (JP)

(72) Inventors: Yuki KUWAJIMA, Decatur, AL (US); Lensey SMITH, Decatur, AL (US); Keisuke HAGI, Orangeburg, NY (US); Takeshi INABA, Osaka (JP); Hiroaki WADA, Osaka (JP)

(73) Assignees: DAIKIN AMERICA INC., Orangeburg, NY (US); DAIKIN INDUSTRIES, LTD., Osaka (JP)

(21) Appl. No.: 19/644,061

(22) Filed: Apr. 10, 2026

Related U.S. Application Data
(63) Continuation-in-part of application No. PCT/IB2024/059856, filed on Oct. 9, 2024.
(60) Provisional application No. 63/543,832, filed on Oct. 12, 2023.

(51) Int. Cl. A61L 2/26; A61L 2/08; A61L 101/48

(52) U.S. Cl. CPC A61L 2/26; A61L 2/08; A61L 2101/48

(57) ABSTRACT

Bioprocess bags are provided which retain good mechanical characteristics after radiation sterilization. The bags are constructed from a copolymer of tetrafluoroethylene. The tetrafluoroethylene is polymerized with at least one monomer selected from the group consisting of: ethylene; chlorotrifluoroethylene; vinyl fluoride; hexafluoropropylene; hexafluoroisobutene; monomers represented by CH₂═CX¹(CF₂)ₙX² (wherein X¹ is H or F, X² is H, F, or Cl, and n is an integer of 1 to 20); perfluoro(alkyl vinyl ethers) represented by CF₂═CF—ORf¹ (wherein Rf¹ is a C1-C20 perfluoroalkyl group or a C1-C17 perfluoroalkoxy alkyl group); and alkyl perfluorovinyl ether derivatives represented by CF₂═CF—OCH₂—Rf² (wherein Rf² is a C1-C5 perfluoroalkyl group). A particular version is polymerized with ethylene and CH₂═CX¹(CF₂)ₙX² (wherein X¹ is H or F, X² is H, F, or Cl, and n is an integer of 1 to 20). Various versions of the bag have high resistance to repeated bending, folding, and creasing; low water vapor transmissibility; high puncture resistance; high biocompatibility; and low reactivity. This makes them suitable for storing biomaterials and medical materials.

### CROSS-REFERENCE TO RELATED APPLICATIONS

This application is a continuation-in-part of International Application No. PCT/IB2024/059856 filed Oct. 9, 2024, which claims priority based on U.S. Provisional Patent Application No. 63/543,832, filed Oct. 12, 2023, the respective disclosures of which are incorporated herein by reference in their entirety.

### TECHNICAL FIELD

The present disclosure relates generally to sterilizable bags for processing and storing biological, biochemical, and medical materials. Such bags, as well as methods of using them and making them, are described in this disclosure.

### BACKGROUND ART

The recent pandemic of SARS-CoV-2 virus vividly illustrated the critical importance of the medical supply chain in modern society. Biological and biochemical technologies, such as antiserum, mRNA, and antibody probes, are of great value in addressing public health problems. However, these types of materials are often unstable at ambient temperatures, requiring extremely low temperature storage. They are also vulnerable to microbial and viral contamination, which requires sterilization of their containers. The same is also generally true of other medical materials.

Both sterilization and cryogenic temperatures can have a negative effect on the mechanical and chemical characteristics of polymer materials. Although non-polymeric materials, such as metals and ceramics, can be used with biological and biochemical materials (or “biomaterials”) and medical materials (such as drugs), they tend to be impractical and expensive. Polymeric materials that are conventionally used as containers become brittle and delicate at cryogenic temperatures or after exposure to sterilizing radiation. There is need in the art for a flexible container that has high resistance to stress cracking and low water vapor permeability after sterilization by ionizing radiation. There also is need in the art for a flexible container that has high resistance to stress cracking and low water vapor permeability under cryogenic conditions.

### SUMMARY

The present disclosure describes a bioprocess bag that addresses the problems described above, although it is to be understood that not all embodiments of the bag will address every such problem. In this context a “bioprocess bag” is a bag for containing a biomaterial or medical material, as further elaborated below.

Various embodiments provide one or more of the following novel and advantageous functions after sterilization by irradiation and/or exposure to cryogenic temperatures: resistance to repeated bending, folding, and creasing; low water vapor transmissibility; high puncture resistance; high biocompatibility; and low reactivity.

Bags are provided in this disclosure that address one or more of these problems by virtue of being constructed from a copolymer of tetrafluoroethylene(TFE). The tetrafluoroethylene is polymerized with at least one monomer selected from the group consisting of: ethylene; chlorotrifluoroethylene; vinyl fluoride; hexafluoropropylene; hexafluoroisobutene; monomers represented by CH₂═CX¹(CF₂)ₙX² (wherein X¹ is H or F, X² is H, F, or Cl, and n is an integer of 1 to 20); perfluoro(alkyl vinyl ethers) represented by CF₂═CF—ORf¹ (wherein Rf¹ is a C1-C20 perfluoroalkyl group or a C1-C17 perfluoroalkoxy alkyl group); and alkyl perfluorovinyl ether derivatives represented by CF₂═CF—OCH₂—Rf² (wherein Rf² is a C1-C5 perfluoroalkyl group).

A first general embodiment of the bioprocess bag is formulated to have enhanced resistance to repeated bending, folding, and creasing.

A second general embodiment of the bioprocess bag is formulated to have enhanced resistance to water vapor transmission.

A third general embodiment of the bioprocess bag is formulated such that the third monomer has an n value that is an integer from 4-10.

A fourth general embodiment of the bioprocess bag is formulated such that the third monomer is at least about 1.7% mol/mol of the collective mole of the monomers.

A method making a bioprocess bag is provided. Embodiments of the method described below address one or more of the problems discussed above using a film constructed from ETFE that includes the third monomer of Formula (I) that is formed into a bag, for example by heat-sealing.

In a first general embodiment of the method of making the bioprocess bag, the resulting bag has enhanced resistance to repeated bending, folding, and creasing.

In a second general embodiment of the method of making the bioprocess bag, the resulting bag has enhanced resistance to water vapor transmission.

In a third general embodiment of the method of making the bioprocess bag, the third monomer has an n value that is an integer from 4-10.

In a fourth general embodiment of the method of making the bioprocess bag, the third monomer is at least about 1.7% mol/mol of the collective mole of the monomers.

A bioprocess bag that is the product of any of the processes described herein is provided. Embodiments of the bioprocess bag that is the product of any of the processes described herein may have one or more of: enhanced resistance to repeated bending, folding, and creasing; low water vapor transmissibility; high puncture resistance; high biocompatibility; and low reactivity.

A method of sterilizing a bioprocess bag is provided, comprising exposing any of the bioprocess bags described herein to a level of ionizing radiation sufficient to sterilize the bioprocess bag. A sterile bioprocess bag that is the product of the process of sterilizing is provided, wherein the bioprocess bag is sterile. Embodiments of the sterilized bioprocess bag that is the product of the process of sterilization may have one or more of: enhanced resistance to repeated bending, folding, and creasing; low water vapor transmissibility; high puncture resistance; high biocompatibility; and low reactivity.

A method of storing a biomaterial or medical material using the bioprocess bag is provided. A general embodiment of the method of storing a biomaterial comprises enclosing the biomaterial or medical material in a sterile bioprocess bag, and storing the biomaterial in the bag for at least 24 hours.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### DESCRIPTION OF EMBODIMENTS

### Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this detailed description are approximate unless stated otherwise, meaning that the term “about” or “approximately” can be inferred when not expressly stated. Numerical quantities in the claims should not be assumed to be approximate, unless the context clearly indicates otherwise. The presence or absence of such terms of approximation should not be interpreted as limiting what is (or is not) equivalent to anything claimed.

With reference to the use of the word(s) “comprise,” “comprises,” and “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “including” should be interpreted to mean “including but not limited to” unless the context clearly indicate otherwise.

The term “consisting essentially of” means that, in addition to the recited elements, what is claimed may also contain other elements (steps, structures, ingredients, components, etc.) that do not adversely affect the operability of what is claimed for its intended purpose. Such addition of other elements that do not adversely affect the operability of what is claimed for its intended purpose would not constitute a material change in the basic and novel characteristics of what is claimed.

The term “adapted to” means designed or configured to accomplish the specified objective, not simply able to be made to accomplish the specified objective.

The term “capable of” means able to be made to accomplish the specified objective.

The terminology used herein is for the purpose of describing particular embodiments only and is not intended to be limiting. As used herein, the singular forms “a”, “an” and “the” are intended to include the plural forms as well (i.e. “at least one”), unless the context clearly indicates otherwise.

The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

Terms such as “at least one of A and B” should be understood to mean “only A, only B, or both A and B.” The same construction should be applied to longer list (e.g., “at least one of A, B, and C”).

None of the definitions above are intended to define what might be considered “equivalent” to anything that is claimed, under the “doctrine of equivalents” or analogous laws.

### Copolymers

The subject matter described in this application employs fluorinated copolymers to impart desirable characteristics to the bioprocess bags.

In a general embodiment, the fluorinated copolymer is a copolymer of TFE. The tetrafluoroethylene is polymerized with at least one monomer selected from the group consisting of: ethylene; chlorotrifluoroethylene; vinyl fluoride; hexafluoropropylene; hexafluoroisobutene; monomers represented by CH₂═CX¹(CF₂)ₙX² (wherein X¹ is H or F, X² is H, F, or Cl, and n is an integer of 1 to 20); perfluoro(alkyl vinyl ethers) represented by CF₂═CF—ORf¹ (wherein Rf¹ is a C1-C20 perfluoroalkyl group or a C1-C17 perfluoroalkoxy alkyl group); and alkyl perfluorovinyl ether derivatives represented by CF₂═CF—OCH₂—Rf² (wherein Rf² is a C1-C5 perfluoroalkyl group).

In a preferred embodiment, TFE is polymerized with ethylene. In such embodiments the copolymer is referred to as an ETFE, although it is understood that other monomers will be incorporated into the copolymer.

In the polymerization, tetrafluoroethylene (TFE) is preferably polymerized with at least one monomer selected from the group consisting of ethylene (Et); chlorotrifluoroethylene (CTFE); vinyl fluoride; hexafluoropropylene (HFP); hexafluoroisobutene; monomers represented by CH₂═CX¹(CF₂)ₙX² (wherein X¹ is H or F, X² is H, F, or Cl, and n is an integer of 1 to 20); perfluoro(alkyl vinyl ethers) (PAVE) represented by CF₂═CF—ORf¹ (wherein Rf¹ is a C1-C20 perfluoroalkyl group or a C1-C17 perfluoroalkoxy alkyl group, preferably a C5-C20 perfluoroalkyl group or a C4-C17 perfluoroalkoxy alkyl group); and alkyl perfluorovinyl ether derivatives represented by CF₂═CF—OCH₂—Rf² (wherein Rf² is a C1-C5 perfluoroalkyl group). In particular, the TFE is more preferably polymerized with at least one monomer selected from the group consisting of ethylene; chlorotrifluoroethylene; hexafluoropropylene; monomers represented by CH₂═CX¹(CF₂)ₙX² (wherein X¹ is H or F, X² is H, F, or Cl, and n is an integer of 1 to 20); and perfluoro(alkyl vinyl ethers). A monomer to be copolymerized with tetrafluoroethylene is preferably at least one selected from the group consisting of ethylene, chlorotrifluoroethylene, perfluoro(alkyl vinyl ethers), and hexafluoropropylene. A particularly preferred combination is a combination of tetrafluoroethylene and ethylene, or a combination of tetrafluoroethylene, ethylene, and hexafluoropropylene.

The polymerization is preferably performed by emulsion polymerization, suspension polymerization, solution polymerization, or bulk polymerization.

In a preferred embodiment, the copolymer is formed from a third monomer that is a fluoropolymer. A preferred example of the third monomer has the formula CH₂═CX¹(CF₂)ₙX², wherein X¹ and X² are each an independently selected F, Cl, or H; and n is an integer of 1 to 20. Another preferred example has the formula CH₂═CX¹(CF₂)ₙCF₂X² (wherein X¹ is H or F, X² is H, F, or Cl, and n is an integer of 0 to 20).

In some embodiments of the third monomer, X¹ is selected from a halide or hydrogen; and X² is H, F, or Cl. In some embodiments of the third monomer, X¹ is selected from a halide or hydrogen; and X² is H or F. In some embodiments of the third monomer, X¹ is selected from a halide or hydrogen; and X² is H or Cl. In some embodiments of the third monomer, X¹ is selected from a halide or hydrogen; and X² is F or Cl. In some embodiments of the third monomer, X¹ is selected from a halide or hydrogen; and X² is H. In some embodiments of the third monomer, X¹ is selected from a halide or hydrogen; and X² is F. In some embodiments of the third monomer, X¹ is selected from a halide or hydrogen; and X² is Cl.

In some embodiments of the third monomer, X² is selected from a halide or hydrogen; and X¹ is H, F, or Cl. In some embodiments of the third monomer, X² is selected from a halide or hydrogen; and X¹ is H or F. In some embodiments of the third monomer, X² is selected from a halide or hydrogen; and X¹ is H or Cl. In some embodiments of the third monomer, X² is selected from a halide or hydrogen; and X¹ is F or Cl. In some embodiments of the third monomer, X² is selected from a halide or hydrogen; and X¹ is H. In some embodiments of the third monomer, X² is selected from a halide or hydrogen; and X¹ is F. In some embodiments of the third monomer, X² is selected from a halide or hydrogen; and X¹ is Cl.

In a further preferred embodiment, X¹ is H or F; X² is H, F, or Cl; and n is an integer from 1-10.

Specific embodiments of the third monomer are: CH₂═CH(CF₂)H; CH₂═CH(CF₂)F; CH₂═CH(CF₂)Cl; CH₂═CF(CF₂)H; CH₂═CF(CF₂)F; CH₂═CF(CF₂)Cl; CH₂═CCl(CF₂)H; CH₂═CCl(CF₂)F; CH₂═CCl(CF₂)Cl; CH₂═CH(CF₂)₂H; CH₂═CH(CF₂)₂F; CH₂═CH(CF₂)₂Cl; CH₂═CF(CF₂)₂H; CH₂═CF(CF₂)₂F; CH₂═CF(CF₂)₂Cl; CH₂═CCl(CF₂)₂H; CH₂═CCl(CF₂)₂F; CH₂═CCl(CF₂)₂Cl; CH₂═CH(CF₂)₃H; CH₂═CH(CF₂)₃F; CH₂═CH(CF₂)₃Cl; CH₂═CF(CF₂)₃H; CH₂═CF(CF₂)₃F; CH₂═CF(CF₂)₃Cl; CH₂═CCl(CF₂)₃H; CH₂═CCl(CF₂)₃F; CH₂═CCl(CF₂)₃Cl; CH₂═CH(CF₂)₄H; CH₂═CH(CF₂)₄F; CH₂═CH(CF₂)₄Cl; CH₂═CF(CF₂)₄H; CH₂═CF(CF₂)₄F; CH₂═CF(CF₂)₄Cl; CH₂═CCl(CF₂)₄H; CH₂═CCl(CF₂)₄F; CH₂═CCl(CF₂)₄Cl; CH₂═CH(CF₂)₅H; CH₂═CH(CF₂)₅F; CH₂═CH(CF₂)₅Cl; CH₂═CF(CF₂)₅H; CH₂═CF(CF₂)₅F; CH₂═CF(CF₂)₅Cl; CH₂═CCl(CF₂)₅H; CH₂═CCl(CF₂)₅F; CH₂═CCl(CF₂)₅Cl; CH₂═CH(CF₂)₆H; CH₂═CH(CF₂)₆F; CH₂═CH(CF₂)₆Cl; CH₂═CF(CF₂)₆H; CH₂═CF(CF₂)₆F; CH₂═CF(CF₂)₆Cl; CH₂═CCl(CF₂)₆H; CH₂═CCl(CF₂)₆F; CH₂═CCl(CF₂)₆Cl; CH₂═CH(CF₂)₇H; CH₂═CH(CF₂)₇F; CH₂═CH(CF₂)₇Cl; CH₂═CF(CF₂)₇H; CH₂═CF(CF₂)₇F; CH₂═CF(CF₂)₇Cl; CH₂═CCl(CF₂)₇H; CH₂═CCl(CF₂)₇F; CH₂═CCl(CF₂)₇Cl; CH₂═CH(CF₂)₈H; CH₂═CH(CF₂)₈F; CH₂═CH(CF₂)₈Cl; CH₂═CF(CF₂)₈H; CH₂═CF(CF₂)₈F; CH₂═CF(CF₂)₈Cl; CH₂═CCl(CF₂)₈H; CH₂═CCl(CF₂)₈F; CH₂═CCl(CF₂)₈Cl; CH₂═CH(CF₂)₉H; CH₂═CH(CF₂)₉F; CH₂═CH(CF₂)₉Cl; CH₂═CF(CF₂)₉H; CH₂═CF(CF₂)₉F; CH₂═CF(CF₂)₉Cl; CH₂═CCl(CF₂)₉H; CH₂═CCl(CF₂)₉F; CH₂═CCl(CF₂)₉Cl; CH₂═CH(CF₂)₁₀H; CH₂═CH(CF₂)₁₀F; CH₂═CH(CF₂)₁₀Cl; CH₂═CF(CF₂)₁₀H; CH₂═CF(CF₂)₁₀F; CH₂═CF(CF₂)₁₀Cl; CH₂═CCl(CF₂)₁₀H; CH₂═CCl(CF₂)₁₀F; and CH₂═CCl(CF₂)₁₀Cl.

In some preferred embodiments of the third monomer, X¹ is H and X² is F. In further preferred embodiments, X¹ is F and X² is H. In some overlapping preferred embodiments of the third monomer, n is an integer from 2-10, 2-9, 2-8, 2-7, 2-6, 2-5, 2-4, and 2-3. In further preferred embodiments, n is 1. In further preferred embodiments, n is 2. In further preferred embodiments, n is 3. In further preferred embodiments, n is 4. In further preferred embodiments, n is 5. In further preferred embodiments, n is 6. In further preferred embodiments, n is 7. In further preferred embodiments, n is 8. In further preferred embodiments, n is 9. In further preferred embodiments, n is 10. In further preferred embodiments, n is at least 2. In further preferred embodiments, n is at least 3. In further preferred embodiments, n is at least 4. In further preferred embodiments, n is at least 5. In further preferred embodiments, n is at least 6. In further preferred embodiments, n is at least 7. In further preferred embodiments, n is at least 8. In further preferred embodiments, n is at least 9. In further preferred embodiments, n is up to 2. In further preferred embodiments, n is up to 3. In further preferred embodiments, n is up to 4. In further preferred embodiments, n is up to 5. In further preferred embodiments, n is up to 6. In further preferred embodiments, n is up to 7. In further preferred embodiments, n is up to 8. In further preferred embodiments, n is up to 9.

In specific embodiments of the copolymer, the third monomer is selected from: CH₂═CF(CF₂)₃H, CH₂═CH(CF₂)₅CF₃, and CH₂═CH(CF₂)₃CF₃.

The third monomer may be present at a mole ratio compared to the other monomers that is sufficient to confer improved material performance, including enhanced resistance to repeated bending, folding, and creasing; low water vapor transmissibility; high puncture resistance; high biocompatibility; low reactivity; and any combination of two or more of the foregoing. For example, the third monomer may be about 1% to about 4% mol/mol of the collective mole of the monomers. In further embodiments, the third monomer may be at least 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, or 3.1% mol/mol of the collective mole of the monomers. In further embodiments, the third monomer may be at most 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, or 3.2% mol/mol of the collective mole of the monomers. In specific embodiments, the third monomer may be 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, or 3.2% mol/mol of the collective mole of the monomers. In a preferred embodiment, the third monomer is 1.7-4.0% mol/mol of the collective mole of the monomers. In a further preferred embodiment, the third monomer is 2.0-3.5% mol/mol of the collective mole of the monomers. In a further preferred embodiment, the third monomer is 2.25-3.25% mol/mol of the collective mole of the monomers.

Preferred formulations of the composition having CH₂═CF(CF₂)₃H as the third monomer may include at least 1.7% mol/mol CH₂═CF(CF₂)₃H of the collective mole of the monomers. Further preferred formulations include at least 1.8, 1.9, or 2.0% mol/mol CH₂═CF(CF₂)₃H of the collective mole of the monomers. Some preferred formulations contain no more than 4% mol/mol CH₂═CF(CF₂)₃H of the collective mole of the monomers. Further preferred embodiments contain no more than 3.5% mol/mol CH₂═CF(CF₂)₃H of the collective mole of the monomers. Further preferred embodiments contain no more than 2.8% mol/mol CH₂═CF(CF₂)₃H of the collective mole of the monomers. Further preferred embodiments contain 1.7-4.0%, 1.8-3.8%, 2.0-3.5%, 1.9-2.9%, 2.4%, or 3.2% mol/mol CH₂═CF(CF₂)₃H of the collective mole of the monomers.

Preferred formulations of the composition having CH₂═CH(CF₂)₅CF₃, CH₂═CH(CF₂)₃CF₃, or both, may include at least 0.5% mol/mol CH₂═CH(CF₂)₅CF₃, CH₂═CH(CF₂)₃CF₃, or both, of the collective mole of the monomers. Further preferred formulations of the composition having CH₂═CH(CF₂)₅CF₃, CH₂═CH(CF₂)₃CF₃, or both, may include at least 0.6, 0.7, 0.8, 0.9, 1.0, 1.1, 1.2, or 1.3% mol/mol CH₂═CH(CF₂)₅CF₃, CH₂═CH(CF₂)₃CF₃, or both, of the collective mole of the monomers. Some preferred formulations of the composition having CH₂═CH(CF₂)₅CF₃, CH₂═CH(CF₂)₃CF₃, or both, may include at most 2.5% mol/mol CH₂═CH(CF₂)₅CF₃, CH₂═CH(CF₂)₃CF₃, or both, of the collective mole of the monomers. Further preferred formulations of the composition having CH₂═CH(CF₂)₅CF₃, CH₂═CH(CF₂)₃CF₃, or both, may include at most 2.4, 2.3, 2.2, 2.1, 2.0, 1.9, 1.8, 1.7, 1.6, 1.5, 1.4, 1.3, % mol/mol CH₂═CH(CF₂)₅CF₃, CH₂═CH(CF₂)₃CF₃, or both, of the collective mole of the monomers. Further preferred formulations of the composition having CH₂═CH(CF₂)₅CF₃, CH₂═CH(CF₂)₃CF₃, or both, may include 0.5-5.0, 0.5-3.0, or 1.0-2.0% mol/mol CH₂═CH(CF₂)₅CF₃, CH₂═CH(CF₂)₃CF₃, or both, of the collective mole of the monomers.

Some embodiments of the copolymer comprise exactly three monomers. Further embodiments of the copolymer comprise more than one type of monomer as described above for the third monomer. Further embodiments of the copolymer comprise a fourth monomer that is co-polymerizable with both ethylene and tetrafluoroethylene.

Examples of the fourth monomer copolymerizable with both TFE and Et include monomers represented by any of the following formulas: CH₂═CX₃Rf³, CF₂═CFRf³, CF₂═CFORf³, and CH₂═C(Rf³)₂ wherein X³ is a hydrogen atom or a fluorine atom, and Rf³ is a fluoroalkyl group which may optionally have: an oxygen atom which serves as an ether bond, a carbonyl oxygen or carbonyl oxygens, or an acid anhydride oxygen. In particular, the fourth monomer is preferably at least one selected from the group consisting of fluorovinyl monomers represented by any of CF₂═CFRf³, CF₂═CFORf³, and CH₂═CX³Rf³, and more preferably at least one selected from the group consisting of HFP; perfluoro(alkyl vinyl ethers) represented by CF₂═CFORf⁴ (wherein Rf⁴ is a C1-C20 perfluoroalkyl group or a C1-C17 perfluoroalkoxy alkyl group); and fluorovinyl monomers represented by CH₂═CX³Rf⁵ (wherein Rf⁵ is a C1-C8 fluoroalkyl group). Examples of the fluorovinyl monomers include CH₂═CH—C₄F₉, CH₂═CF—CF₂—CF₂—CF₂H, CH₂═CH—C₆F₁₃, and CH₂═CH—C₈F₁₅.

### Bioprocess Bags

The copolymers described above find use in the construction of bioprocess bags. The bioprocess bags are intended to contain biomaterials and medical materials, and have excellent mechanical characteristics after exposure to sterilizing levels of ionizing radiation. This can involve a level of radiation sufficient to achieve at least six-log kill of a particular pathogen. Examples of sterilizing levels of ionizing radiation are 50, 300, and 600 kGy.

Preferred embodiments of the bioprocess bag may have enhanced resistance to repeated bending, folding, and creasing over ETFE alone. Such enhanced resistance may be measured by any suitable method, including standard methods such as the MIT Folding Endurance Test (ASTM D2176-16 (2021)), which is incorporated by reference into this disclosure insofar as necessary to clearly define what is claimed. Some preferred embodiments of the bag score at least 10,000 on the MIT Folding Endurance Test (ASTM D2176-16 (2021)) after exposure to 50 kGy of γ-radiation. Further preferred embodiments of the bag score at least 10⁴, 1.2×10⁴, 1.4×10⁴, 1.6×10⁴, 1.8×10⁴, 2.0×10⁴, 2.5×10⁴, 3.0×10⁴, 3.5×10⁴, 4.0×10⁴, 4.5×10⁴, 5.0×10⁴, 1.0×10⁵, 1.5×10⁵, or 2.0×10⁵ on the MIT Folding Endurance Test (ASTM D2176-16 (2021)) after exposure to 50 kGy of γ-radiation.

Preferred embodiments of the bag may have enhanced resistance to water vapor transmission over ETFE alone. Such enhanced resistance may be measured by any suitable method, including standard methods such as ASTM F1249-13 (2013), which is incorporated by reference into this disclosure insofar as necessary to clearly define what is claimed. Some preferred embodiments of the bag display a water vapor transmissibility rate of no more than 6.0 g-mil per m² per 24 h by ASTM F1249-13 (2013) after exposure to 50 kGy of γ-radiation. Further preferred embodiments of the bag display a water vapor transmissibility rate of no more than 5.9, 5.8, 5.7, 5.6, 5.5, 5.4, 5.3, 5.2, 5.1, 5.0, 4.9, 4.8, 4.7, 4.6, 4.5, 4.4, or 4.3 g-mil per m² per 24 h by ASTM F1249-13 (2013) after exposure to 50 kGy of γ-radiation.

Preferred embodiments of the bag may have enhanced puncture resistance over ETFE alone. Such enhanced resistance may be measured by any suitable method, including standard methods such as ASTM D3763-18 (2018) performed at −70 degrees C., which is incorporated by reference into this disclosure insofar as necessary to clearly define what is claimed. Some preferred embodiments of the bag display a puncture resistance of at least 2800 J at −70° C. as measured by ASTM D3763-18 (2018) after exposure to 50 kGy of γ radiation. Further preferred embodiments of the bag display a puncture resistance of at least 2900 J at −70° C. as measured by ASTM D3763-18 (2018) after exposure to 50 kGy of γ radiation.

Preferred embodiments of the bag may have enhanced tear strength over ETFE alone. Such enhanced tear strength may be measured by any suitable method, including standard methods such as ASTM D1004-21 (2021), which is incorporated by reference into this disclosure insofar as necessary to clearly define what is claimed. Some preferred embodiments of the bag display a tear strength of at least 650 as measured by ASTM D1004-21 (2021) after exposure to 50 kGy of γ-radiation.

Some embodiments of the bioprocess bag comprise a layer that is at least 50% mol/mol of the copolymer. In further embodiments, the layer comprises at least 55, 60, 65, 70, 75, 80, 85, 90, 91, 92, 93, 94, 95, 96, 97, 98, 99, or 100% mol/mol of the copolymer. In a specific embodiment of the method, the layer consists essentially of the copolymer.

Preferred embodiments of the bioprocess bag are adapted for, or configured to, contain a biomaterial, a medical material, or both. Further preferred embodiments of the bag are adapted for, or configured to, be sterilized using ionizing radiation, contain materials at cryogenic temperatures, or both.

The bioprocess bag may be the product of the method of making bioprocess bags described below. It may also be a sterile bioprocess bag, that is the product of the method of sterilization described below.

### Methods of Making Bioprocess Bags

The bioprocess bags described herein may be made by heat-sealing a film to form the bag. The film comprises any embodiment of the copolymer described above. In some embodiments of the bag, the film comprises at least 55, 60, 65, 70, 75, 80, 85, 90, 91, 92, 93, 94, 95, 96, 97, 98, 99, or 100% mol/mol of the copolymer. In a specific embodiment of the method, the film consists essentially of the copolymer.

### Methods of Sterilization

A method of sterilizing a bioprocess bag is disclosed. The method involves exposing any of the bioprocess bags described herein to a sterilizing level of ionizing radiation.

Preferred forms of ionizing radiation for this purpose are V-rays and X-rays. The amount of radiation should be enough to at least significantly reduce the number of viable microorganisms present, if not eliminate them altogether. Embodiments of the method employ levels of radiation sufficient to produce at least a 6-log kill of microorganisms. Further embodiments employ levels of radiation sufficient to produce at least a 7, 8, or 9-log kill of microorganisms. In a preferred embodiment of the method the bag is exposed to at least 50 kGy of ionizing radiation, such as V-rays and X-rays. In further embodiments of the method higher levels of radiation may be used, such as 300 kGy or 600 kGy. Some embodiments of the method involve exposure of the bag to 50-600 kGy of ionizing radiation.

### Methods of Storing Materials in Bioprocess Bags

A method of storing materials in a bioprocess bag is provided. The bags are intended to store biomaterials and medical materials. The bags could also be used to store any material that benefits from sterile storage, storage at cryogenic temperatures, or both. The material is enclosed in the bag, and then stored. The period of storage may be protracted. Examples of suitable storage periods are at least 0.5 hours. Further examples are at least 1, 2, 4 h, 6 h, 8 h, 12 h, 24 h, 48 h, 72 h, 96 h, one week, two weeks, one month, two months, three months, four months, six months, and twelve months.

Some embodiments of the bag have excellent structural characteristics when stored at low temperatures. Thus, in some embodiments of the method, the bag and material are stored below 0° C. In further embodiments of the method, the bag and material are stored at or below −17, −28, −40, −54, −60, −70, or −80° C.

The bag may contain a suitable biomaterial. Biomaterials must generally remain sterile, and many degrade unless stored at low temperature. Examples of such biomaterials include a tissue, a bodily fluid, blood, serum, plasma, an isolated nucleic acid, an isolated protein, an antibiotic, an amplification mixture, a culture medium, a cell, a microorganism, an organism, an organ, an isolated antibody, an isolated enzyme, a vaccine, an embryo, a zygote, or any combination of two or more of the foregoing.

The bag can also be used to contain a medical material. Like biomaterials, medical materials must generally remain sterile. Some are not stable for long unless stored at low temperature. Examples of medical materials that could be stored in the bag include: a drug, a biologic agent, a saline solution, an electrolyte solution, a glucose solution, a buffer, a diluent, a cryoprotectant, an anticoagulant, a dialysis replacement fluid, a dialysate, a vitamin solution, a mineral solution, an enteral nutrition liquid, or any combination of two or more of the foregoing. Note that some medical materials are also biomaterials (and vice-versa), and the two characterizations of materials are not mutually exclusive.

### Examples

Films were made by copolymerizing TFE and ethylene with a third monomer chosen from CH₂═CF(CF₂)₃H, CH₂═CH(CF₂)₅CF₃, and CH₂═CH(CF₂)₃CF₃. The relative amounts in moles of the monomers in each film are shown below in Table 1.

TABLE 1

| Test # | 1 | 2 | 3 | 4 | 5 |
|---|---|---|---|---|---|
| TFE | 63.4 | 62.9 | 54.2 | 54.2 | 56.3 |
| Ethylene | 34.2 | 33.9 | 44.3 | 44.4 | 42.4 |
| CH₂═CF(CF₂)₃H | 2.4 | 3.2 | 1.5 | | |
| CH₂═CH(CF₂)₃CF₃ | | | | 1.4 | |
| CH₂═CH(CF₂)₅CF₃ | | | | | 1.3 |

The properties of each film were measured after exposure to four different amounts of γ-irradiation and after exposure to 100 kGy of X-radiation. Water vapor transmissibility (WVTR) was measured according to ASTM F1249-13 (2013). Puncture resistance was measured at −70° C. according to ASTM D3763-18 (2018). Tear strength was measured according to ASTM D1004-21 (2021). Resistance to repeated bending, folding, and creasing was measured according to the MIT Folding Endurance Test (ASTM D2176-16 (2021)), using films having a thickness of 250 μm. Results of testing are shown in Tables 2 and 3.

TABLE 2

| | Test | (kGy) γ-irradiation | | | |
|---|---|---|---|---|---|
| Test # | Test contents | 0 | 50 | 300 | 600 |
| 1 | WVTR | 4.22 | 4.43 | 3.86 | 3.92 |
| 2 | (g-mil/m²/24 hr) | 5.67 | 5.83 | 6.08 | 5.99 |
| 3 | | 4.9 | 4.77 | 4.46 | 4.52 |
| 4 | | 5.14 | 5.06 | 4.65 | 4.49 |
| 5 | | 4.44 | 4.28 | 4.04 | 4.36 |
| 1 | Puncture Resistance | 2840 | 2910 | 2780 | 215 |
| 2 | (J) | 3070 | 3060 | 364 | 364 |
| 3 | | 3630 | 3730 | 3710 | 277 |
| 4 | | 3450 | 3790 | 3600 | 247 |
| 5 | | 3480 | 3387 | 3380 | 239 |
| 1 | Tear Strength | 688 | 692 | 493 | 473 |
| 2 | (lbf/in) | 653 | 673 | 618 | 511 |
| 3 | | 1123 | 1095 | 844 | 736 |
| 4 | | 995 | 1020 | 790 | 710 |
| 5 | | 956 | 967 | 733 | 650 |
| 1 | MIT | 167836 | 48731 | 500 | 24 |
| 2 | | 994454 | 242385 | 576 | 89 |
| 3 | | 7966 | 3819 | 226 | 48 |
| 4 | | 26072 | 17047 | 416 | 41 |
| 5 | | 14562 | 8246 | 496 | 108 |

TABLE 3

| | |
|---|---|
| Test | (kGy) X-irradiation |

| | Test contents | 0 | 100 |
|---|---|---|---|
| 1 | WVTR | 4.22 | |
| 2 | (g-mil/m²/24 hr) | 5.67 | |
| 3 | | 4.9 | |
| 4 | | 5.14 | |
| 5 | | 4.44 | |
| 1 | Puncture Resistance | 2840 | |
| 2 | N = 2 + 1 | 3070 | |
| 3 | (J) | 3630 | |
| 4 | | 3450 | |
| 5 | | 3480 | |
| 1 | Tear Strength | 688 | 688 |
| 2 | (lbf/in) | 653 | 735 |
| 3 | | 1123 | 1016 |
| 4 | | 995 | 980 |
| 5 | | 956 | 944 |
| 1 | MIT | 167836 | 1493 |
| 2 | | 994454 | 9644 |
| 3 | | 7966 | 2052 |
| 4 | | 26072 | 1358 |
| 5 | | 14562 | 859 |

### CONCLUSIONS

It is to be understood that any given elements of the disclosed embodiments of the invention may be embodied in a single structure, a single step, a single substance, or the like. Similarly, a given element of the disclosed embodiment may be embodied in multiple structures, steps, substances, or the like. The foregoing description and accompanying drawings illustrate and describe certain processes, machines, manufactures, and compositions of matter, some of which embody the invention(s). Such descriptions or illustrations are not intended to limit the scope of what can be claimed, and are provided as aids in understanding the claims, enabling the making and use of what is claimed, and teaching the best mode of use of the invention(s). If this description and accompanying drawings are interpreted to disclose only a certain embodiment or embodiments, it shall not be construed to limit what can be claimed to that embodiment or embodiments. Any examples or embodiments of the invention described herein are not intended to indicate that what is claimed must be coextensive with such examples or embodiments. Where it is stated that the invention(s) or embodiments thereof achieve one or more objectives, it is not intended to limit what can be claimed to versions capable of achieving all such objectives. Any statements in this description criticizing the prior art are not intended to limit what is claimed to exclude any aspects of the prior art. Additionally, the disclosure shows and describes certain embodiments of the processes, machines, manufactures, compositions of matter, and other teachings disclosed, but it is to be understood that the teachings of the present disclosure are capable of use in various other combinations, modifications, and environments and is capable of changes or modifications within the scope of the teachings as expressed herein. Any section headings herein are provided only for consistency with the suggestions of 37 C.F.R. § 1.77 or otherwise to provide organizational queues. These headings shall not limit or characterize the invention(s) set forth herein.

The following is claimed:

1. A bioprocess bag constructed from a copolymer formed from the following monomers: tetrafluoroethylene, ethylene, and a third monomer having the formula 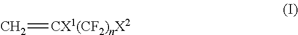
wherein
X¹ is H or F
X² is H, F, or Cl and
n is an integer from 1-20;
and wherein the bioprocess bag scores at least 10,000 on the MIT Folding Endurance Test (ASTM D2176-16 (2021)) after exposure to 50 kGy of γ-radiation.

2. A bioprocess bag constructed from a copolymer formed from the following monomers: tetrafluoroethylene, ethylene, and a third monomer having the formula 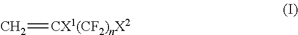
wherein
X¹ is H or F
X² is H, F, or Cl and
n is an integer from 1-20;
and wherein the bioprocess bag scores at least 10,000 on the MIT Folding Endurance Test (ASTM D2176-16 (2021)) and displays a water vapor transmissibility rate of no more than 5.5 g-mil per m² per 24 h by ASTM F1249-13 (2013) after exposure to 50 kGy of γ-radiation.

3. A bioprocess bag constructed from a copolymer formed from the following monomers: tetrafluoroethylene, ethylene, and a third monomer having the formula 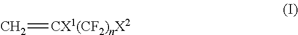
wherein
X¹ is H or F
X² is H, F, or Cl
n is an integer from 4-10.

4. A bioprocess bag constructed from a copolymer formed from the following monomers: tetrafluoroethylene, ethylene, and a third monomer having the formula 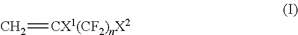
wherein
X¹ is H or F
X² is H, F, or Cl and
n is an integer from 1-20;
wherein the third monomer is at least about 1.7% mol/mol of collective mole of the monomers.

5. A method of making a bioprocess bag, the method comprising:
(a) providing a fluoropolymer film of a copolymer formed from the following monomers: tetrafluoroethylene, ethylene, and a third monomer having the formula 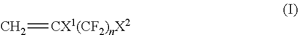
wherein
X¹ is H or F
X² is H, F, or Cl and
n is an integer from 1-20; and
(b) heating-sealing the film to form the bag;
wherein the bioprocess bag scores at least 10,000 on the MIT Folding Endurance Test (ASTM D2176-16 (2021)) after exposure to 50 kGy of γ-radiation.

6. A method of making a bioprocess bag, the method comprising:
(a) providing a fluoropolymer film of a copolymer formed from the following monomers: tetrafluoroethylene, ethylene, and a third monomer having the formula 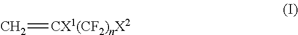
wherein
X¹ is H or F
X² is H, F, or Cl and
n is an integer from 1-10;
wherein the third monomer is at least about 1.7% mol/mol of collective mole of the monomers; and
(b) heating-sealing the film to form the bag.

7. The bioprocess bag that is the product of any one of the methods of claims 5-6.

8. Any one of the bioprocess bags or methods above, wherein X¹ is H and X² is F.

9. Any one of the bioprocess bags or methods above, wherein the third monomer is selected from:

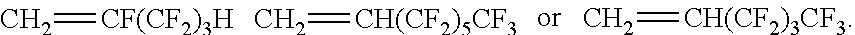

10. Any one of the bioprocess bags or methods above, wherein the third monomer has the formula CH₂═CF(CF₂)₃H, and is 1.7-4.0% mol/mol of the collective mole of the monomers.

11. Any one of the bioprocess bags or methods above, wherein the third monomer is selected from CH₂═CH(CF₂)₅CF₃, CH₂═CH(CF₂)₃CF₃, or both; and is 0.5-5.0% mol/mol of the collective mole of the monomers.

12. Any one of the bioprocess bags or methods above, wherein the bioprocess bag has a puncture resistance of at least 2800 J at −70° C. as measured by ASTM D3763-18 (2018) after exposure to 50 kGy of γ radiation; and wherein the bioprocess bag scores at least 10,000 on the MIT Folding Endurance Test (ASTM D2176-16 (2021)) after exposure to 50 kGy of γ-radiation.

13. Any one of the bioprocess bags or methods above, wherein the bioprocess bag is configured to contain a biomaterial or medical material.

14. Any one of the bioprocess bags or methods above, wherein the bioprocess bag is configured to be sterilized by ionizing radiation.

15. Any one of the bioprocess bags or methods above, wherein the bioprocess bag is configured to store a material at cryogenic temperatures.

16. A method of sterilizing a bioprocess bag, comprising: exposing any one of the bioprocess bags of the claims above to at least 50 kGy of ionizing radiation.

17. A sterile bioprocess bag that is the product of the process of claim 16.

18. A method of storing a biological or biochemical material, comprising: enclosing the material in the sterile bioprocess bag of claim 17; and storing the material and the bag for at least 24 hours.

19. The method of storing a biological or biochemical material of claim 18, wherein the material and the bag are stored at a temperature about equal to or less than −54° C.

20. The method of storing a biological or biochemical material of claim 18, wherein the biological material contains: a tissue, a bodily fluid, blood, serum, plasma, an isolated nucleic acid, an isolated protein, an antibiotic, an amplification mixture, a culture medium, a cell, a microorganism, an organ, an isolated antibody, an isolated enzyme, a vaccine, an embryo, a zygote, or a combination of two or more of the foregoing.
